(19) United States
(12) Patent Application Publication
(10) Pub. No.: US 2023/0397946 A1
(43) Pub. Date: Dec. 14, 2023

(54) END EFFECTOR ASSEMBLY WITH MULTI-AXIS THERMAL CUTTING ELEMENT

(71) Applicant: Covidien LP, Mansfield, MA (US)

(72) Inventors: James D. Allen, IV, Broomfield, CO (US); William E. Robinson, Boulder, CO (US); Daniel A. Joseph, Golden, CO (US); John A. Hammerland, III, Arvada, CO (US); Kenneth E. Netzel, Loveland, CO (US)

(21) Appl. No.: 18/032,985

(22) PCT Filed: Oct. 5, 2021
(86) PCT No.: PCT/US2021/053463

§ 371 Date: Apr. 20, 2023

Related U.S. Application Data
(60) Provisional application No. 63/093,937, filed on Oct. 20, 2020.

(51) Int. Cl. A61B 18/14; A61B 18/12; A61B 18/08

(52) U.S. Cl. CPC A61B 18/1442; A61B 18/1206; A61B 18/082; A61B 2018/00601

(57) ABSTRACT

An end effector assembly for an electrosurgical instrument includes a pair of opposing jaw members each including a jaw housing supporting an electrically conductive tissue engaging surface thereon. The jaw members are movable relative to one another to grasp tissue therebetween. A thermal cutting element is operatively associated with the electrically conductive tissue engaging surface of one jaw member and is independently activatable relative to the electrically conductive tissue engaging surfaces. The thermal cutting element is selectively moveable in a first direction relative to the electrically conductive tissue engaging surface along a transverse axis perpendicular to a longitudinal axis defined through the jaw member and is selectively moveable in a second direction along the longitudinal axis to extend relative to a distal end of the jaw member.

### CROSS-REFERENCE TO RELATED APPLICATION

The present application claims priority to U.S. Provisional Patent Application No. 63,093,937, filed on Oct. 20, 2020.

### FIELD

The present disclosure relates to surgical instruments and, more particularly, to thermal cutting elements, electrosurgical instruments including thermal cutting elements, and methods of manufacturing thermal cutting elements.

### BACKGROUND

A surgical forceps is a pliers-like instrument that relies on mechanical action between its jaw members to grasp, clamp, and constrict tissue. Electrosurgical forceps utilize both mechanical clamping action and energy to heat tissue to treat, e.g., coagulate, cauterize, or seal, tissue. Typically, once tissue is treated, the surgeon has to accurately sever the treated tissue. Accordingly, many electrosurgical forceps are designed to incorporate a knife that is advanced between the jaw members to cut the treated tissue. As an alternative to a mechanical knife, an energy-based tissue cutting element may be provided to cut the treated tissue using energy, e.g., thermal, electrosurgical, ultrasonic, light, or other suitable energy.

### SUMMARY

As used herein, the term “distal” refers to the portion that is being described which is further from a user, while the term “proximal” refers to the portion that is being described which is closer to a user. Further, to the extent consistent, any or all of the aspects detailed herein may be used in conjunction with any or all of the other aspects detailed herein.

Provided in accordance with aspects of the present disclosure is end effector assembly for an electrosurgical instrument that includes a pair of jaw members each including a jaw housing supporting an electrically conductive tissue engaging surface thereon. The jaw members oppose one another on opposite sides of a longitudinal axis defined through one of the jaw members. The electrically conductive tissue engaging surfaces are disposed in opposition relative to one another. One (or both) of the pair of jaw members is movable relative to the other of the pair of jaw members to grasp tissue therebetween. The electrically conductive tissue engaging surfaces are adapted to connect to an electrosurgical energy source.

A thermal cutting element is operatively associated with one (or both) of the electrically conductive tissue engaging surfaces. The thermal cutting element is independently activatable relative to the electrically conductive tissue engaging surfaces and is adapted to connect to the electrosurgical energy source. The thermal cutting element is exposed along a length of the electrically conductive tissue engaging surface of the jaw member and is selectively moveable in a first direction relative to the electrically conductive tissue engaging surface along a transverse axis perpendicular to the longitudinal axis. The thermal cutting element is selectively moveable in a second direction along the longitudinal axis to extend relative to a distal end of the jaw member.

In aspects according to the present disclosure, the end effector assembly cooperates with an actuator configured to move the thermal cutting element in the first direction. In other aspects according to the present disclosure, the end effector assembly cooperates with an actuator configured to move the thermal cutting element in the second direction. In yet other aspects according to the present disclosure, the end effector assembly cooperates with an actuator configured to move the thermal cutting element in the first or second direction. In still other aspects according to the present disclosure, the actuator includes a T-channel actuator configured to move the thermal cutting element in either the first and second directions.

In aspects according to the present disclosure, the thermal cutting element cooperates with a switch configured to selectively energize the thermal cutting element upon activation thereof. In aspects according to the present disclosure, the thermal cutting element is activated based upon selected generator inputs or outputs or based on a thermal cutting element algorithm.

In aspects according to the present disclosure, the thermal cutting element is movably supported within an insulative material disposed in the electrically conductive tissue engaging surface of the jaw member. In other aspects according to the present disclosure, the insulative material is a thermally insulative material, an electrically insulative material or an electrically and thermally insulative material.

Provided in accordance with other aspects of the present disclosure is a surgical instrument that includes a housing having a shaft extending from a distal end thereof. A distal end of the shaft is configured to support an end effector assembly thereon, the end effector assembly including a pair of jaw members each including a jaw housing supporting an electrically conductive tissue engaging surface thereon. The jaw members opposing one another on opposite sides of a longitudinal axis defined therethrough, the electrically conductive tissue engaging surfaces are disposed in opposition relative to one another. One (or both) of the pair of jaw members is movable relative to the other of the pair of jaw members to grasp tissue therebetween. The electrically conductive tissue engaging surfaces are adapted to connect to an electrosurgical energy source.

A thermal cutting element is operatively associated with the electrically conductive tissue engaging surface of one (or both) of the jaw members. The thermal cutting element is independently activatable relative to the electrically conductive tissue engaging surfaces and is adapted to connect to the electrosurgical energy source. The thermal cutting element is exposed along a length of the electrically conductive tissue engaging surface and is selectively moveable in a first direction relative to the electrically conductive tissue engaging surface along a transverse axis perpendicular to the longitudinal axis. The thermal cutting element is also selectively moveable in a second direction along the longitudinal axis to extend relative to a distal end of the jaw member.

In aspects according to the present disclosure, an actuator is included on the housing and is configured to move the thermal cutting element in the first or the second direction. In other aspects according to the present disclosure, the actuator includes a T-channel actuator configured to move the thermal cutting element in either the first and second directions.

In aspects according to the present disclosure, a switch is disposed on the housing and is configured to selectively energize the thermal cutting element upon activation thereof.

In aspects according to the present disclosure, the thermal cutting element is movably supported within an insulative material disposed in the electrically conductive tissue engaging surface of the at least one jaw member. In other aspects according to the present disclosure, the insulative material is a thermally insulative material, an electrically insulative material or an electrically and thermally insulative material.

Provided in accordance with other aspects of the present disclosure is a surgical instrument that includes a pair of first and second shaft members each including a handle disposed at a proximal end thereof and a jaw member disposed at a distal end thereof. The shaft members are selectively moveable relative to one another to move the jaw members about a pivot from a first spaced apart position to a second position for approximating tissue. An electrically conductive tissue engaging surface is disposed on each jaw member and is adapted to connect to an electrosurgical energy source.

A thermal cutting element is operatively associated with one (or both) of the electrically conductive tissue engaging surfaces of one of the jaw members. The thermal cutting element is independently activatable relative to the electrically conductive tissue engaging surfaces and is adapted to connect to the electrosurgical energy source. The thermal cutting element is exposed along a length of the electrically conductive tissue engaging surface of the jaw member. The thermal cutting element is selectively moveable in a first direction relative to the electrically conductive tissue engaging surface along a transverse axis perpendicular to a longitudinal axis defined through the jaw member. The thermal cutting element is selectively moveable in a second direction along the longitudinal axis to extend relative to a distal end of the jaw member.

In aspects according to the present disclosure, an actuator is disposed on one of the first or second shaft members and is configured to move the thermal cutting element in the first or the second direction. In other aspects according to the present disclosure, the actuator is a T-channel actuator configured to move the thermal cutting element in either the first and second directions.

In aspects according to the present disclosure, a switch disposed on one (or both) of the shaft members and is configured to selectively energize the thermal cutting element upon activation thereof.

In aspects according to the present disclosure, the thermal cutting element is movably supported within an insulative material disposed in the electrically conductive tissue engaging surface of the at least one jaw member. In other aspects according to the present disclosure, the insulative material is a thermally insulative material, an electrically insulative material or an electrically and thermally insulative material.

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1 is a perspective view of a shaft-based electrosurgical forceps provided in accordance with the present disclosure shown connected to an electrosurgical generator;

FIG. 2 is a perspective view of a hemostat-style electrosurgical forceps provided in accordance with the present disclosure;

FIG. 3 is a schematic illustration of a robotic surgical instrument provided in accordance with the present disclosure;

FIG. 4 is a perspective view of a distal end portion of the forceps of FIG. 1, wherein first and second jaw members of an end effector assembly of the forceps are disposed in a spaced-apart position;

FIG. 5A is a bottom, perspective view of the first jaw member of the end effector assembly of FIG. 4;

FIG. 5B is a top, perspective view of the second jaw member of the end effector assembly of FIG. 4;

FIG. 6A is a schematic, side view of an end effector including an embodiment of a thermal cutting element according to the present disclosure; and

FIG. 6B is a schematic, top view of the end effector and thermal cutting element of FIG. 6A.

### DETAILED DESCRIPTION

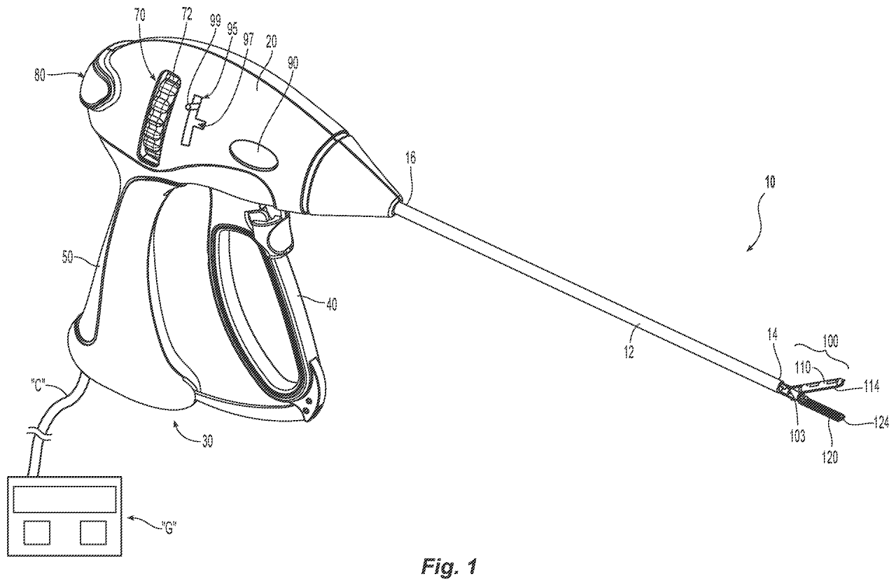

Referring to FIG. 1, a shaft-based electrosurgical forceps provided in accordance with the present disclosure is shown generally identified by reference numeral 10. Aspects and features of forceps 10 not germane to the understanding of the present disclosure are omitted to avoid obscuring the aspects and features of the present disclosure in unnecessary detail.

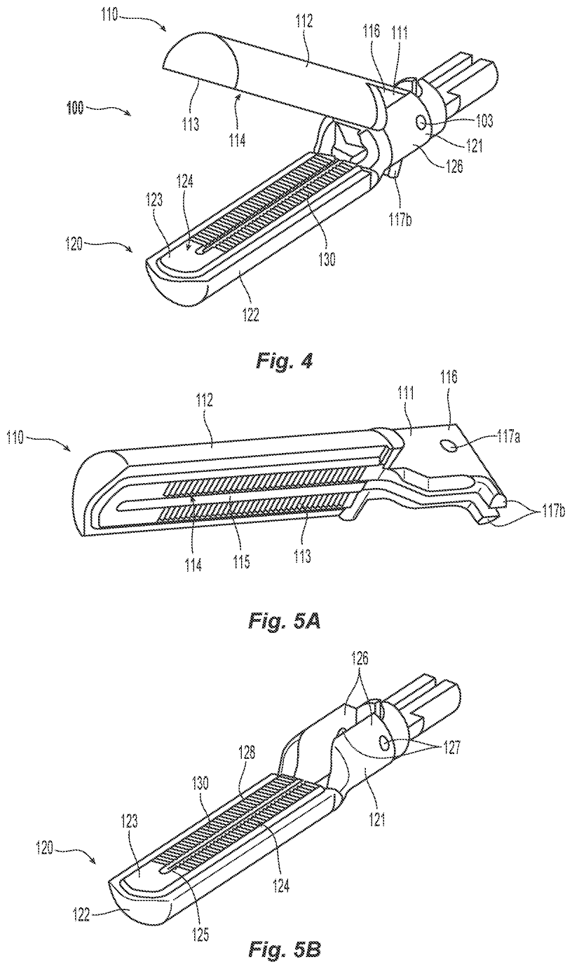

Forceps 10 includes a housing 20, a handle assembly 30, a trigger assembly 60, a rotating assembly 70, a first activation switch 80, a second activation switch 90, and an end effector assembly 100. Forceps 10 further includes a shaft 12 having a distal end portion 14 configured to (directly or indirectly) engage end effector assembly 100 and a proximal end portion 16 that (directly or indirectly) engages housing 20. Forceps 10 also includes cable “C” that connects forceps 10 to an energy source, e.g., an electrosurgical generator “G.” Cable “C” includes a wire (or wires) (not shown) extending therethrough that has sufficient length to extend through shaft 12 in order to connect to one or both tissue-treating surfaces 114, 124 of jaw members 110, 120, respectively, of end effector assembly 100 (see FIG. 4) to provide energy thereto. First activation switch 80 is coupled to tissue-treating surfaces 114, 124 (FIG. 4) and the electrosurgical generator “G” for enabling the selective activation of the supply of energy to jaw members 110, 120 for treating, e.g., cauterizing, coagulating/desiccating, and/or sealing, tissue. Second activation switch 90 is coupled to thermal cutting element 130 of jaw member 120 (FIG. 4) and the electrosurgical generator “G” for enabling the selective activation of the supply of energy to thermal cutting element 150 for thermally cutting tissue.

Handle assembly 30 of forceps 10 includes a fixed handle 50 and a movable handle 40. Fixed handle 50 is integrally associated with housing 20 and handle 40 is movable relative to fixed handle 50. Movable handle 40 of handle assembly 30 is operably coupled to a drive assembly (not shown) that, together, mechanically cooperate to impart movement of one or both of jaw members 110, 120 of end effector assembly 100 about a pivot 103 between a spaced-apart position and an approximated position to grasp tissue between tissue-treating surfaces 114, 124 of jaw members 110, 120. As shown in FIG. 1, movable handle 40 is initially spaced-apart from fixed handle 50 and, correspondingly, jaw members 110, 120 of end effector assembly 100 are disposed in the spaced-apart position. Movable handle 40 is depressible from this initial position to a depressed position corresponding to the approximated position of jaw members 110, 120. Rotating assembly 70 includes a rotation wheel 72 that is selectively rotatable in either direction to correspondingly rotate end effector assembly 100 relative to housing 20.

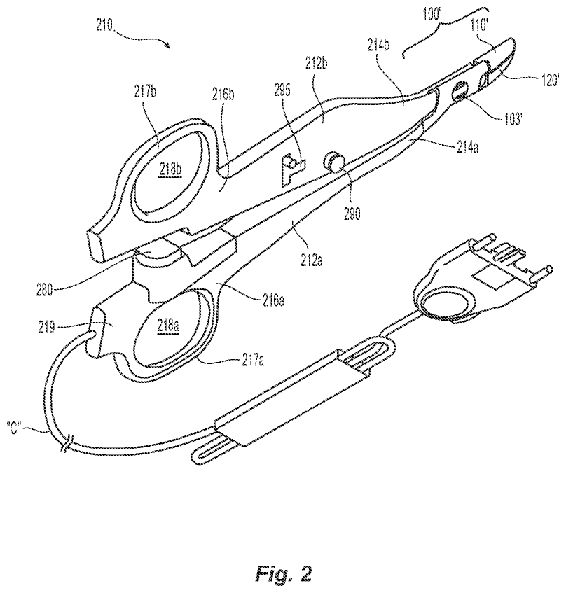

Referring to FIG. 2, a hemostat-style electrosurgical forceps provided in accordance with the present disclosure is shown generally identified by reference numeral 210. Aspects and features of forceps 210 not germane to the understanding of the present disclosure are omitted to avoid obscuring the aspects and features of the present disclosure in unnecessary detail.

Forceps 210 includes two elongated shaft members 212a, 212b, each having a proximal end portion 216a, 216b, and a distal end portion 214a, 214b, respectively. Forceps 210 is configured for use with an end effector assembly 100′ similar to end effector assembly 100 (FIG. 4). More specifically, end effector assembly 100′ includes first and second jaw members 110′, 120′ attached to respective distal end portions 214a, 214b of shaft members 212a, 212b. Jaw members 110′, 120′ are pivotably connected about a pivot 103′. Each shaft member 212a, 212b includes a handle 217a, 217b disposed at the proximal end portion 216a, 216b thereof. Each handle 217a, 217b defines a finger hole 218a, 218b therethrough for receiving a finger of the user. As can be appreciated, finger holes 218a, 218b facilitate movement of the shaft members 212a, 212b relative to one another to, in turn, pivot jaw members 110′, 120′ from the spaced-apart position, wherein jaw members 110′, 120′ are disposed in spaced relation relative to one another, to the approximated position, wherein jaw members 110′, 120′ cooperate to grasp tissue therebetween.

One of the shaft members 212a, 212b of forceps 210, e.g., shaft member 212b, includes a proximal shaft connector 219 configured to connect forceps 210 to a source of energy, e.g., electrosurgical generator “G” (FIG. 1). Proximal shaft connector 219 secures a cable “C” to forceps 210 such that the user may selectively supply energy to jaw members 110′, 120′ for treating tissue. More specifically, a first activation switch 280 is provided for supplying energy to jaw members 110′, 120′ to treat tissue upon sufficient approximation of shaft members 212a, 212b, e.g., upon activation of first activation switch 280 via shaft member 212a. A second activation switch 290 disposed on either or both of shaft members 212a, 212b is coupled to the thermal cutting element (not shown, similar to thermal cutting element 150 of jaw member 120 (FIG. 4)) of one of the jaw members 110′, 120′ of end effector assembly 100′ and to the electrosurgical generator “G” for enabling the selective activation of the supply of energy to the thermal cutting element for thermally cutting tissue. It is envisioned that a single switch, e.g., switch 280, may be utilized to activate both the jaw members 110′, 120′ and the thermal cutting element 150 or the thermal cutting element 150 may be activated automatically based on generator input or output parameters or upon completion of the seal cycle. Alternatively, a thermal cutting element algorithm may be employed to automatically activate the thermal cutting element 150.

Jaw members 110′, 120′ define a curved configuration wherein each jaw member is similarly curved laterally off of a longitudinal axis of end effector assembly 100′. However, other suitable curved configurations including curvature towards one of the jaw members 110, 120′ (and thus away from the other), multiple curves with the same plane, and/or multiple curves within different planes are also contemplated. Jaw members 110, 120 of end effector assembly 100 (FIG. 1) may likewise be curved according to any of the configurations noted above or in any other suitable manner.

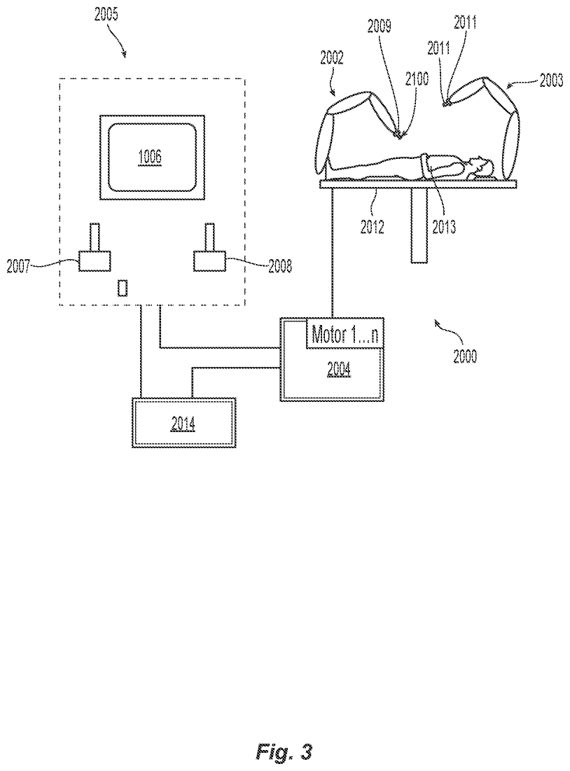

Referring to FIG. 3, a robotic surgical instrument provided in accordance with the present disclosure is shown generally identified by reference numeral 2000. Aspects and features of robotic surgical instrument 2000 not germane to the understanding of the present disclosure are omitted to avoid obscuring the aspects and features of the present disclosure in unnecessary detail.

Robotic surgical instrument 2000 includes a plurality of robot arms 2002, 2003; a control device 2004; and an operating console 2005 coupled with control device 2004. Operating console 2005 may include a display device 2006, which may be set up in particular to display three-dimensional images; and manual input devices 2007, 2008, by means of which a surgeon may be able to telemanipulate robot arms 2002, 2003 in a first operating mode. Robotic surgical instrument 2000 may be configured for use on a patient 2013 lying on a patient table 2012 to be treated in a minimally invasive manner. Robotic surgical instrument 2000 may further include a database 21014, in particular coupled to control device 2004, in which are stored, for example, pre-operative data from patient 2013 and/or anatomical atlases.

Each of the robot arms 2002, 2003 may include a plurality of members, which are connected through joints, and an attaching device 2009, 2011, to which may be attached, for example, an end effector assembly 2100, 2200, respectively. End effector assembly 2100 is similar to end effector assembly 100 (FIG. 4), although other suitable end effector assemblies for coupling to attaching device 2009 are also contemplated. End effector assembly 2200 may be any end effector assembly, e.g., an endoscopic camera, other surgical tool, etc. Robot arms 2002, 2003 and end effector assemblies 2100, 2200 may be driven by electric drives, e.g., motors, that are connected to control device 2004. Control device 2004 (e.g., a computer) may be configured to activate the motors, in particular by means of a computer program, in such a way that robot arms 2002, 2003, their attaching devices 2009, 2011, and end effector assemblies 2100, 2200 execute a desired movement and/or function according to a corresponding input from manual input devices 2007, 2008, respectively. Control device 2004 may also be configured in such a way that it regulates the movement of robot arms 2002, 2003 and/or of the motors.

Turning to FIG. 4, one embodiment of a known end effector assembly 100, as noted above, includes first and second jaw members 110, 120. Each jaw member 110, 120 may include a structural frame 111, 121, a jaw housing 112, 122, and a tissue-treating plate 113, 123 defining the respective tissue-treating surface 114, 124 thereof. Alternatively, only one of the jaw members, e.g., jaw member 120, may include the structural frame 121, jaw housing 122, and tissue-treating plate 123 defining the tissue-treating surface 124. In such embodiments, the other jaw member, e.g., jaw member 110, may be formed as a single unitary body, e.g., a piece of conductive material acting as the structural frame 111 and jaw housing 112 and defining the tissue-treating surface 114. An outer surface of the jaw housing 112, in such embodiments, may be at least partially coated with an insulative material or may remain exposed. For the purposes herein, the term insulative is defined to mean that the thermal or electrical conductivity feature is lower than the surrounding features.

In embodiments, tissue-treating plates 113, 123 may be deposited onto jaw housings 112, 122 or jaw inserts (not shown) disposed within jaw housings 112, 122, e.g., via sputtering. Alternatively, tissue-treating plates 113, 123 may be pre-formed and engaged with jaw housings 112, 122 and/or jaw inserts (not shown) disposed within jaw housings 112, 122 via, for example, overmolding, adhesion, mechanical engagement, etc.

Referring in particular to FIGS. 4-5B, jaw member 110, as noted above, may be configured similarly as jaw member 120, may be formed as a single unitary body, or may be formed in any other suitable manner so as to define a structural frame 111 and a tissue-treating surface 114 opposing tissue-treating surface 124 of jaw member 120. Structural frame 111 includes a proximal flange portion 116 about which jaw member 110 is pivotably coupled to jaw member 120. In shaft-based or robotic embodiments, proximal flange portion 116 may further include an aperture 117a for receipt of pivot 103 and at least one protrusion 117b extending therefrom that is configured for receipt within an aperture defined within a drive sleeve of the drive assembly (not shown) such that translation of the drive sleeve, e.g., in response to actuation of movable handle 40 (FIG. 1) or a robotic drive, pivots jaw member 110 about pivot 103 and relative to jaw member 120 between the spaced-apart position and the approximated position. However, other suitable drive arrangements are also contemplated, e.g., using cam pins and cam slots, a screw-drive mechanism, etc.

Regardless of the particular configuration of jaw member 110, jaw member 110 may include a longitudinally-extending insulative member 115 extending along at least a portion of the length of tissue-treating surface 114. Insulative member 115 may be transversely centered on tissue-treating surface 114 or may be offset relative thereto. Further, insulative member 115 may be disposed, e.g., deposited, coated, etc., on tissue-treating surface 114, may be positioned within a channel or recess defined within tissue-treating surface 114, or may define any other suitable configuration. Additionally, insulative member 115 may be substantially (within manufacturing, material, and/or use tolerances) coplanar with tissue-treating surface 114, may protrude from tissue-treating surface 114, may be recessed relative to tissue-treating surface 114, or may include different portions that are coplanar, protruding, and/or recessed relative to tissue-treating surface 114. Insulative member 115 may be disposed under the tissue-treating surface 114. Insulative member 115 may be formed from, for example, ceramic, parylene, nylon, PTFE, polybenzimidazole, or other suitable material(s) (including combinations of insulative and non-insulative materials).

With reference to FIGS. 4 and 5B, as noted above, jaw member 120 includes a structural frame 121, a jaw housing 122, and a tissue-treating plate 123 defining the tissue-treating surface 124 thereof. Jaw member 120 further include a thermal cutting element 130. Structural frame 121 defines a proximal flange portion 126 and a distal body portion (not shown) extending distally from proximal flange portion 126. Proximal flange portion 126 is bifurcated to define a pair of spaced-apart proximal flange portion segments that receive proximal flange 111 of jaw member 110 therebetween and define aligned apertures 127 configured for receipt of pivot 103 therethrough to pivotably couple jaw members 110, 120 with one another.

Jaw housing 122 of jaw member 120 is disposed about the distal body portion of structural frame 121, e.g., via overmolding, adhesion, mechanical engagement, etc., and supports tissue-treating plate 123 thereon, e.g., via overmolding, adhesion, mechanical engagement, depositing (such as, for example, via sputtering), etc. Tissue-treating plate 123, as noted above, defines tissue-treating surface 124. A longitudinally-extending slot 125 is defined through tissue-treating plate 123 and is positioned to oppose insulative member 115 of jaw member 110 (FIG. 5A) in the approximated position. Slot 125 may extending through at least a portion of jaw housing 122, a jaw insert (if so provided), and/or other components of jaw member 120 to enable receipt of thermal cutting element 130 at least partially within slot 125.

Thermal cutting element 130, more specifically, is disposed within longitudinally-extending slot 125 such that thermal cutting element 130 opposes insulative member 115 of jaw member 110 (FIG. 5A) in the approximated position. Thermal cutting element 130 may be configured to contact insulative member 115 (FIG. 5A) in the approximated position to regulate or contribute to regulation of a gap distance between tissue-treating surfaces 114, 124 in the approximated position. Alternatively or additionally, one or more stop members (not shown) associated with jaw member 110 and/or jaw member 120 may be provided to regulate the gap distance between tissue-treating surfaces 114, 124 in the approximated position.

Thermal cutting element 130 may be surrounded by an insulative member 128 disposed within slot 125 to electrically isolate thermal cutting element from tissue-treating plate 123. Alternatively or additionally, thermal cutting element 130 may include an insulative coating on at least the sides thereof for similar purposes. Thermal cutting element 130 and insulative member 128 may similarly or differently be substantially (within manufacturing, material, and/or use tolerances) coplanar with tissue-treating surface 124, may protrude from tissue-treating surface 124, may be recessed relative to tissue-treating surface 124, or may include different portions that are coplanar, protruding, and/or recessed relative to tissue-treating surface 124.

In embodiments where end effector assembly 100, or a portion thereof, is curved, longitudinally-extending slot 125 and thermal cutting element 130 may similarly be curved, e.g., wherein longitudinally-extending slot 125 and thermal cutting element 130 (or corresponding portions thereof) are relatively configured with reference to an arc (or arcs) of curvature rather than a longitudinal axis. Thus, the terms longitudinal, transverse, and the like as utilized herein are not limited to linear configurations, e.g., along linear axes, but apply equally to curved configurations, e.g., along arcs of curvature. In such curved configurations, insulating member 115 of jaw member 110 (FIG. 5A) is likewise curved.

Generally referring to FIGS. 1-5B, tissue-treating plates 113, 123 are formed from an electrically conductive material, e.g., for conducting electrical energy therebetween for treating tissue, although tissue-treating plates 113, 123 may alternatively be configured to conduct any suitable energy, e.g., thermal, microwave, light, ultrasonic, etc., through tissue grasped therebetween for energy-based tissue treatment. As mentioned above, tissue-treating plates 113, 123 are coupled to activation switch 80 and electrosurgical generator “G” (FIG. 1) such that energy may be selectively supplied to tissue-treating plates 113, 123 and conducted therebetween and through tissue disposed between jaw members 110, 120 to treat tissue, e.g., seal tissue on either side and extending across thermal cutting element 130.

Thermal cutting element 130, on the other hand, is configured to connect to electrosurgical generator “G” (FIG. 1) and second activation switch 90 to enable selective activation of the supply of energy to thermal cutting element 130 for heating thermal cutting element 130 to thermally cut tissue disposed between jaw members 110, 120, e.g., to cut the sealed tissue into first and second sealed tissue portions. Other configurations including multi-mode switches, other separate switches, etc. may alternatively be provided. Cross reference is made to U.S. Provisional Patent Application Ser. No. 62/952,232 the entire contents of which being incorporated by reference herein.

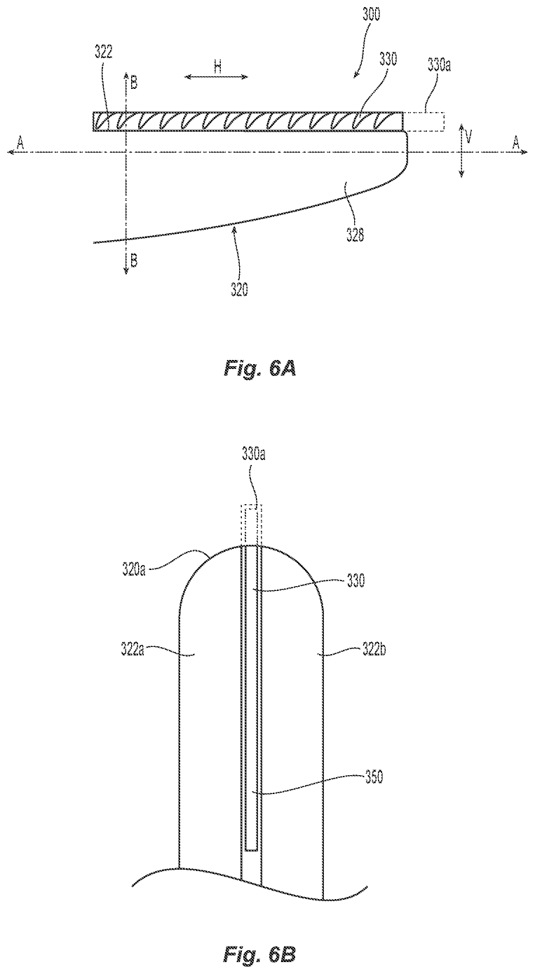

Referring to FIGS. 6A and 6B, another embodiment of a thermal cutting element 330 is shown for use with any of the above-described end effector assemblies and jaw configurations mentioned above. Thermal cutting element 330 is disposed along sealing surface 322 and extends substantially therealong. Thermal cutting element 330 may be centrally located along sealing surface 322 or may be slightly askew depending upon a particular purpose. As shown in FIG. 6B, thermal cutting element 330 is centrally located along sealing surface 322 and is movably supported by an insulative material 350 that is configured to thermally and/or electrically insulate the thermal cutting element 330 from either side 322a, 322b of sealing surface 322.

Thermal cutting element 330 may be individually connected to a generator or other heating source such that thermal cutting element 330 is independently activatable relative to sealing surface 322. Thermal cutting element 330 may also be activated simultaneously or sequentially to maximize sealing during the sealing process if energized with the sealing surface 322 and opposing sealing surface 113 (See FIGS. 1 and 4).

Thermal cutting element 330 is selectively moveable relative to both a longitudinal axis A-A defined through jaw member 320 and a vertical axis B-B defined through jaw member perpendicular to axis A-A. One or more actuators 95, 295 may be disposed on the forceps 10, 210 described above to allow selective deployment of the thermal cutting element 330 as needed. More particularly one type of actuator e.g., a T-channel actuator 95, 295, is shown that allows selective movement of the thermal cutting element along axes A-A or B-B. Automated electrical actuators are also contemplated. A T-shaped channel 97 is defined in the housing 20 and a joy stick-like thumb stick 99 is configured to move in a T-like manner within the channel to move the thermal cutting element 330 along either axis A-A or B-B.

In a first mode or position, the surgeon may opt to move the thermal cutting element 330 vertically along axis B-B to enhance the cutting process. For example, the surgeon may wish to extend the thermal cutting element 330 relative to the plane of the sealing surface to dissect or otherwise treat tissue. Once energized the tissue may be cut or dissected and the cut tissue may slough away on either side of the extended thermal cutting element 330. Moreover, extending the thermal cutting element 330 relative to the sealing surface 322 creates a robust dissection tool for performing a pull and cut dissection technique, e.g., energizing or heating thermal cutting element 330 while pulling the tissue between the jaw members 110 (FIG. 1), 320 away from the remaining tissue. In the first mode or position, the thermal cutting element 330 may also be used to back-score tissue.

The thermal cutting element 330 may also be moved such that the thermal cutting element 330 is flush with the sealing surface 322. Energizing simultaneously or sequentially and with similar power curves may maximize sealing. It is contemplated that the thermal cutting element 330 may be automatically (or manually) moved to a flush orientation and then energized with the same switch 80 during a sealing phase (i.e., once the thermal cutting element 330 is moved to a flush or substantially flush position and switch 80 is activated, the thermal cutting element 330 is also activated to maximize the sealing effect. Thermal cutting element 330 may then be activated with another switch 90 to initiate cutting, the same switch 80 or automatically activated as discussed above.

The thermal cutting element 330 may also be moved such that the thermal cutting element 330 is recessed relative to the sealing surface 322. In situations where sensitive tissue may need to be cut, the surgeon may opt to initially seal tissue between jaw members 110, 320 and then cut in a subsequent step. By recessing the thermal cutting element 330 relative to the sealing surface, heat exchange between the sealing plate 322 and the thermal cutting element 330 is minimized.

As shown in FIGS. 1 and 2, T-channel actuators 95, 295 allow selective movement of the thermal cutting element 330 along the B-B axis. The surgeon may extend, make flush or recess the thermal cutting element 330 as needed prior to, during or after electrical activation of switch 80.

In a second mode or position, the surgeon may opt to move the thermal cutting element 330 horizontally along axis A-A to enhance the cutting or dissection process. More particularly, the surgeon may selectively extend the thermal cutting element 330 such that the distal tip 330a of the thermal cutting element 330 extends beyond the distal end 320a of the jaw member 320. As shown in FIGS. 1 and 2, T-channel actuators 95, 295 may be used to selectively extend the tip 330a as needed prior to, during or after sealing tissue. Joystick and spherical actuators are also envisioned. In addition, the actuator 95, 295 may be continuous or actuate the tip 330a in discreet amounts.

Once the thermal cutting element 330 is energized, tissue may be cut or dissected as the surgeon moves the jaw member 320 distally into tissue. Moreover, extending the distal tip 330 of the thermal cutting element 330 relative to the distal end 320a of the jaw member 320 enables the surgeon to back-score tissue when moving the jaw member 320 proximally along tissue. Once the tissue is treated in a desired fashion, the surgeon can retract the distal tip 330a of the thermal cutting element 330 such that the distal tip 330 is flush with the distal end 320a of the jaw member 320. It is contemplated that the distal tip 330a of the thermal cutting element 330 may also be move to a recessed position relative to the distal end 320a of the jaw member 320. For example, it may be practical in certain instances to recess the distal tip 330 for blunt dissection using the distal end 320a of the jaw member 320.

As mentioned above, switch 90 may be utilized to activate the thermal cutting element 330. Switch 90 may be configured in any fashion to enable activation of thermal cutting element 330 prior to or during a sealing and cutting cycle or for other surgical purposes, e.g., tissue scoring or tissue back-scoring. Switch 90 may include one or more activation elements and may include one or more toggles or rocker switches. A joystick-like switch 90 is also contemplated with different or varying activations. Other known switches are also contemplated.

In embodiments, a second thermal cutting element (not shown) may be disposed on the opposite jaw member, e.g., jaw member 110 (FIG. 1). The second thermal cutting element maybe stationary or move along either or both axes A-A and B-B.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An end effector assembly for an electrosurgical instrument, comprising:
a pair of jaw members each including a jaw housing supporting an electrically conductive tissue engaging surface thereon, the jaw members opposing one another on opposite sides of a longitudinal axis defined therethrough, the electrically conductive tissue engaging surfaces disposed in opposition relative to one another, at least one of the pair of jaw members movable relative to the other of the pair of jaw members to grasp tissue therebetween, the electrically conductive tissue engaging surfaces adapted to connect to an electrosurgical energy source; and
a thermal cutting element operatively associated with at least one of the electrically conductive tissue engaging surfaces, the thermal cutting element independently activatable relative to the electrically conductive tissue engaging surfaces and adapted to connect to the electrosurgical energy source, the thermal cutting element exposed along a length of the at least one electrically conductive tissue engaging surface, the thermal cutting element selectively moveable in a first direction relative to the electrically conductive tissue engaging surface along a transverse axis perpendicular to the longitudinal axis and selectively moveable in a second direction along the longitudinal axis to extend relative to a distal end of the at least one of the pair of jaw members.

2. The end effector assembly according to claim 1, wherein the end effector assembly cooperates with an actuator configured to move the thermal cutting element in the first direction.

3. The end effector assembly according to claim 1, wherein the end effector assembly cooperates with an actuator configured to move the thermal cutting element in the second direction.

4. The end effector assembly according to claim 1, wherein the end effector assembly cooperates with an actuator configured to move the thermal cutting element in the first and second directions.

5. The end effector assembly according to claim 4, wherein the actuator includes a T-channel actuator configured to move the thermal cutting element in either the first and second directions.

6. The end effector assembly according to claim 1, wherein the thermal cutting element cooperates with a switch configured to selectively energize the thermal cutting element upon activation thereof.

7. The end effector assembly according to claim 1, wherein the thermal cutting element is movably supported within an insulative material disposed in the electrically conductive tissue engaging surface of the at least one jaw member.

8. The end effector assembly according to claim 7, wherein the insulative material is a thermally insulative material, an electrically insulative material or an electrically and thermally insulative material.

9. A surgical instrument, comprising:
a housing having a shaft extending from a distal end thereof, a distal end of the shaft supporting an end effector assembly thereon, the end effector assembly including:
a pair of jaw members each including a jaw housing supporting an electrically conductive tissue engaging surface thereon, the jaw members opposing one another on opposite sides of a longitudinal axis defined therethrough, the electrically conductive tissue engaging surfaces disposed in opposition relative to one another, at least one of the pair of jaw members movable relative to the other of the pair of jaw members to grasp tissue therebetween, the electrically conductive tissue engaging surfaces adapted to connect to an electrosurgical energy source; and
a thermal cutting element operatively associated with at least one of the electrically conductive tissue engaging surfaces, the thermal cutting element independently activatable relative to the electrically conductive tissue engaging surfaces and adapted to connect to the electrosurgical energy source, the thermal cutting element exposed along a length of the at least one electrically conductive tissue engaging surface, the thermal cutting element selectively moveable in a first direction relative to the electrically conductive tissue engaging surface along a transverse axis perpendicular to the longitudinal axis and selectively moveable in a second direction along the longitudinal axis to extend relative to a distal end of the at least one of the pair of jaw members.

10. The surgical instrument according to claim 9, further comprising an actuator disposed on the housing configured to move the thermal cutting element in the first or the second direction.

11. The surgical instrument according to claim 10, wherein the actuator includes a T-channel actuator configured to move the thermal cutting element in either the first and second directions.

12. The end effector assembly according to claim 9, further comprising a switch disposed on the housing configured to selectively energize the thermal cutting element upon activation thereof.

13. The end effector assembly according to claim 9, wherein the thermal cutting element is movably supported within an insulative material disposed in the electrically conductive tissue engaging surface of the at least one jaw member.

14. The end effector assembly according to claim 13, wherein the insulative material is a thermally insulative material, an electrically insulative material or an electrically and thermally insulative material.

15. A surgical instrument, comprising:
a pair of first and second shaft members each including a handle disposed at a proximal end thereof and a jaw member disposed at a distal end thereof, the shaft members selectively moveable relative to one another to move the jaw members about a pivot from a first spaced apart position to a second position for approximating tissue;
an electrically conductive tissue engaging surface disposed on each jaw member, each electrically conductive tissue engaging surface adapted to connect to an electrosurgical energy source; and
a thermal cutting element operatively associated with at least one of the electrically conductive tissue engaging surfaces of one of the jaw members, the thermal cutting element independently activatable relative to the electrically conductive tissue engaging surfaces and adapted to connect to the electrosurgical energy source, the thermal cutting element exposed along a length of the electrically conductive tissue engaging surface of the at least one jaw member, the thermal cutting element selectively moveable in a first direction relative to the electrically conductive tissue engaging surface along a transverse axis perpendicular to a longitudinal axis defined through the at least one jaw member and selectively moveable in a second direction along the longitudinal axis to extend relative to a distal end of the at least one jaw member.

16. The surgical instrument according to claim 15, further comprising an actuator disposed on one of the first or second shaft members configured to move the thermal cutting element in the first or the second direction.

17. The surgical instrument according to claim 16, wherein the actuator includes a T-channel actuator configured to move the thermal cutting element in either the first and second directions.

18. The end effector assembly according to claim 15, further comprising a switch disposed on at least one of the shaft members configured to selectively energize the thermal cutting element upon activation thereof.

19. The end effector assembly according to claim 15, wherein the thermal cutting element is movably supported within an insulative material disposed in the electrically conductive tissue engaging surface of the at least one jaw member.

20. The end effector assembly according to claim 19, wherein the insulative material is a thermally insulative material, an electrically insulative material or an electrically and thermally insulative material.
